(19) United States
(12) Patent Application Publication
(10) Pub. No.: US 2004/0116444 A1
(43) Pub. Date: Jun. 17, 2004

(54) Substituted 1,4-pyrazine derivatives

(76) Inventors: Jeffrey W. Corbett, Portage, MI (US); Jian-Min Fu, Kalamazoo, MI (US); Michael Dalton Ennis, Mattawan, MI (US); Kristine E. Frank, Portage, MI (US); Robert Louis Hoffman, Kalamazoo, MI (US); Patrick R. Verhoest, Augusta, MI (US)

Correspondence Address: PHARMACIA & UPJOHN, 301 HENRIETTA ST, 0228-32-LAW, KALAMAZOO, MI, 49007, US

(21) Appl. No.: 10/649,299

(22) Filed: Aug. 27, 2003

Related U.S. Application Data
(60) Provisional application No. 60/410,261, filed on Sep. 12, 2002.

(51) Int. Cl. A61K 31/4965; C 07D 4 3/02

(52) U.S. Cl. 514/255.05; 514/255.06; 544/405; 544/406

(57) ABSTRACT

Substituted aryl 1,4-pyrazine derivatives and their use in treating anxiety disorders, depression and stress related disorders are disclosed.

### CROSS REFERENCE

This application claims the benefit of the following provisional application: U.S. Ser. No 60/410,261, filed Sep. 12, 2002 under 35 USC 119(e)(i), which is incorporated herein by reference in its entirety.

### FIELD OF THE INVENTION

This invention relates to substituted aryl 1,4-pyrazine derivatives and processes for preparing them, pharmaceutical compositions containing them, and methods of using them to treat of anxiety disorders, depression and stress related disorders. The compounds are also useful in smoking cessation programs, certain central nervous system (CNS) disorders, and other disorders. CRF antagonists possess multiple uses including the use of such compounds in the treatment of a disorder or condition which can be effected of facilitated by antagonizing CRF, including but not limited to disorders induced or facilitated by CRF, such as of anxiety disorders, depression and stress related disorders. Additionally this invention relates to the use of such compounds as probes for the localization of CRF₁ receptors in cells and tissues.

### BACKGROUND OF THE INVENTION

Corticotropin releasing factor (CRF) is a 41 amino acid peptide that is the primary physiological regulator of proopiomelanocortin (POMC) derived peptide secretion from the anterior pituitary gland [J. Rivier et al., Proc. Natl. Acad. Sci (USA) 80:4851 (1983); W. Vale et al., Science 213:1394 (1981)]. In addition to its endocrine role at the pituitary gland, immunohistochemical localization of CRF has demonstrated that the hormone has a broad extrahypothalamic distribution in the central nervous system and produces a wide spectrum of autonomic, electrophysiological and behavioral effects consistent with a neurotransmitter or neuromodulator role in the brain [W. Vale et al., Rec. Prog. Horm. Res. 39:245 (1983); G. F. Koob, Persp. Behav. Med. 2:39 (1985); E. B. De Souza et al., J. Neurosci. 5:3189 (1985)]. There is also evidence that CRF plays a significant role in integrating the response in the immune system to physiological, psychological, and immunological stressors [J. E. Blalock, Physiological Reviews 69:1 (1989); J. E. Morley, Life Sci. 41:527 (1987)].

There is evidence that CRF has a role in psychiatric disorders and neurological diseases including depression, anxiety-related disorders and feeding disorders. A role for CRF has also been postulated in the etiology and pathophysiology of Alzheimer's disease, Parkinson's disease, Huntington's disease, progressive supranuclear palsy and amyotrophic lateral sclerosis, as they relate to the dysfunction of CRF neurons in the central nervous system [for a review, see: E. B. De Souze, Hosp. Practice 23:59 (1988)].

Anxiety disorders are a group of diseases, recognized in the art, that includes phobic disorders, anxiety states, posttraumatic stress disorder and atypical anxiety disorders [The Merck Manual of Diagnosis and Therapy, 16^{th} edition (1992)]. Emotional stress is often a precipitating factor in anxiety disorders, and such disorders generally respond to medications that lower response to stress.

In affective disorder, or major depression, the concentration of CRF is significantly increased in the cerebral spinal fluid (CSF) of drug-free individuals [C. B. Nemeroff et al., Science 226:1342 (1984); C. M. Banki et al., Am. J. Psychiatry 144:873 (1987); R. D. France et al., Biol. Psychiatry 28:86 (1988); M. Arato et al., Biol. Psychiatry 25:355 (1989)]. Furthermore, the density of CRF receptors is significantly decreased in the frontal cortex of suicide victims, consistent with a hypersecretion of CRF [C. B. Memeroff et al., Arch. Gen. Psychiatry 45:577 (1988)]. In addition, there is a blunted adrenocorticotropin (ACTH) response to CRF (i.v. administered) observed in depressed patients [P. W. Gold et al., Am. J. Psychiatry 141:619 (1984); F. Holsboer et al., Psychoneuroendocrinology 9:147 (1984); P. W. Gold et al., New Engl. J. Med. 314:1129 (1986)]. Preclinical studies in rats and non-human primates provide additional support for the hypothesis that hypersecretion of CRF may be involved in the symptoms seen in human depression [R. M. Sapolsky, Arch. Gen. Psychiatry 46:1047 (1989)]. There is also preliminary evidence that tricyclic antidepressants can alter CRF levels and thus modulate the numbers of receptors in the brain [Grigoriadis et al., Neuropsychopharmacology 2:53 (1989)].

CRF has also been implicated in the etiology of anxiety-related disorders, and is known to produce anxiogenic effects in animals. Interactions between benzodiazepine/non-benzodiazepine anxiolytics and CRF have been demonstrated in a variety of behavioral anxiety models [D. R. Britton et al., Life Sci. 31:363 (1982); C. W. Berridge and A. J. Dunn Regul. Peptides 16:83 (1986)]. Preliminary studies using the putative CRF receptor antagonist α-helical ovine CRF (9-41) in a variety of behavioral paradigms demonstrates that the antagonist produces “anxiolytic-like” effects that are qualitatively similar to the benzodiazepines [C. W. Berridge and A. J. Dunn Horm. Behav. 21:393 (1987), Brain Research Reviews 15:71 (1990)].

Neurochemical, endocrine and receptor binding studies have all demonstrated interactions between CRF and benzodiazepine anxiolytics, providing further evidence for the involvement of CRF in these disorders. Chlodiazepoxide attenuates the “anxiogenic” effects of CRF both in the conflict test [K. T. Britton et al., Psychopharmacology 86:170 (1985); K. T. Britton et al., Psychopharmacology 94:306 (1988)] and in the acoustic startle test [N. R. Swerdlow et al., Psychopharmacology 88:147 (1986)] in rats. The benzodiazipine receptor antagonist Ro 15-1788, which was without behavioral activity alone in the operant conflict test, reversed the effects of CRF in a dose-dependent manner while the benzodiazepine inverse agonist FG 7142 enhanced the actions of CRF [K. T. Britton et al., Psychopharmacology 94:396 (1988)]. The mechanisms and sites of action through which conventional anxiolytics and antidepressants produce their therapeutic effects remain to be elucidated. Preliminary studies, examining the effects of a CRF receptor antagonist peptide (α-helical CRF₉₋₄₁) in a variety of behavioral paradigms, have demonstrated that the CRF antagonist produces “anxiolytic-like” effects qualitatively similar to the benzodiazepines [for a review, see: G. F. Koob and K. T. Britton, In: Corticotropin-Releasing Factor: Basic and Clinical Studies of a Neuropeptide, E. B. De Souza and C. B. Nemeroff eds., CRC Press p.221 (1990)].

The use of CRF antagonists for the treatment of Syndrome X has also been described in U.S. patent application Ser. No. 09/696,822, filed Oct. 26, 2000, and European Patent Application No. 003094414, filed Oct. 26, 2000, which are also incorporated in their entireties herein by reference. Methods for using CRF antagonists to treat congestive heart failure are described in U.S. Ser. No. 09/248,073, filed Feb. 10, 1999, now U.S. Pat. No. 6,043,260 (Mar. 28, 2000) which is also incorporated herein in its entirety by reference.

CRF is known to have a broad extrahypothalmic distribution in the CNS, contributing therein to a wide spectrum of autonomic behavioral and physiological effects [see, e.g., Vale et al., 1983; Koob, 985; and E. B. De Souze et al., 1985]. For example, CRF concentrations are significantly increased in the cerebral spinal fluid of patients afflicted with affective disorder or major depression [see, e.g., Nemeroff et al., 1984; Banki et al., 1987; France et al., 1988; Arato et al., 1989]. Moreover, excessive levels of CRF are known to produce anxiogenic effects in animal models [see, e.g., Britton et al., 1982; Berridge and Dunn, 1986 and 1987], and, CRF antagonists are known to produce anxiolytic effects; accordingly, therapeutically effective amounts of compounds provided herein are, for example, determined by assessing the anxiolytic effects of varying amounts of the compounds in such animal models.

WO 01/60806 discloses aryl piperazines compounds that can bind with high affinity and high selectivity to CRF₁ receptors. The compounds are useful for treating CNS-related disorders particularly affective disorders and diseases, and acute and chronic neurological disorders and diseases.

### SUMMARY OF THE INVENTION

The invention provides compounds of the Formula I as well as stereoisomers and pharmaceutically acceptable salts and prodrugs thereof, which interact with CRF₁ receptors. It further relates to the use of such compounds, pharmaceutical compositions comprising these compounds and methods useful for the treatment of psychiatric and affective disorders and neurological diseases involving CRF₁ receptors.

Surprisingly we have found that compounds of Formula I are CRF antagonists and are useful in the treatment of anxiety disorders, depression and stress related disorders. The compounds are also useful in smoking cessation programs.

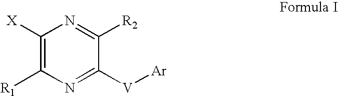

X is selected from —NR₃R₄, —OR₃, —CR₃R₅R₅, —C(O)R₃, —S(O)ₘR₃, —NR₃C(O)R₄, —NR₃S(O)ₘR₄;

V is selected from —O—, —NR₅, or —S(O)ₘ;

m is 0, 1 or 2;

R₁ and R₂ are independently selected from —NH(alkyl), —N(alkyl)₂, —NH(substituted alkyl), —N(substituted alkyl)₂, —O(alkyl), —O(substituted alkyl), halogen, alkyl, substituted alkyl, haloalkyl, cycloalkyl, substituted cycloalkyl, substituted phenyl, naphthyl, substituted naphthyl, heteroaryl, heteroaryl derivatives, substituted aryl, heterocycloalkyl, substituted heterocycloalkyl, substituted heteroaryl, —CR₅R₆Ar, —OAr, —S(O)ₘAr, —NR₅Ar, —S(O)ₘalkyl, —S(O)ₘsubstituted alkyl, —NO₂, —OH, —NH₂, —SH, —C(O)NR₄R₅, —C(S)NR₄R₅, —C(O)NR₅Ar, —S(O)ₘNR5Ar, —NR₅C(O)Ar, —NR₅S(O)nAr, —C(O)Ar, -(alkyl linker)S(O)ₘNH₂, -(alkyl linker)S(O)ₘNR₅Ar, and -(alkyl linker)C(O)Ar;

R₃ and R₄ are independently selected from —H, alkyl, substituted alkyl, haloalkyl, cycloalkyl, substituted cycloalkyl, aryl, heterocycloalkyl, substituted heterocycloalkyl, substituted heteroaryl, aryl cycloalkyl, substituted aryl cycloalkyl, heteroaryl cycloalkyl, substituted heteroaryl cycloalkyl, aryl heterocycloalkyl, substituted aryl heterocycloalkyl, heteroaryl heterocycloalkyl, or substituted heteroaryl heterocycloalkyl;

Each R₅ is independently selected from —H, alkyl, alkylene, alkylyne, cycloalkyl, haloalkyl, and alkyl substituted with 1-3 substituents selected from halogen, —O(alkyl), —NH(alkyl), —N(alkyl)₂, —C(O)NH(alkyl), —C(O)N(alkyl)₂, —NHC(O)alkyl, —N(alkyl)C(O)alkyl, —S(O)ₘalkyl, heterocycloalkyl, substituted heterocycloalkyl and Ar.

Each R₆ is independently selected from alkyl, cycloalkyl, haloalkyl, and alkyl substituted with 1-3 substituents selected from halogen, —O(alkyl), —NH(alkyl), —N(alkyl)₂, —C(O)NH(alkyl), —C(O)N(alkyl)₂, —NHC(O)alkyl, —N(alkyl)C(O)alkyl, —S(O)ₘalkyl, heterocycloalkyl, substituted heterocycloalkyl and Ar;

Halogen is a group selected from —F, —Cl, —Br, —I;

Alkyl means both straight- and branched chain hydrocarbon chains having from 1-10 carbon atoms;

Alkylene means both straight- and branched chain hydrocarbon chains having from 2-10 carbon atoms and a double bond;

Alkylyne means both straight- and branched chain hydrocarbon chains having from 2-10 carbon atoms and a triple bond;

Substituted alkyl is an alkyl moiety from 1-10 carbon atoms having 1-3 substituents independently selected from halogen, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, CN, —NO₂, and Ar;

Haloalkyl is an alkyl moiety having from 1-10 carbon atoms and having 1 to (2v+1) independently selected halogen substituent(s) where v is the number of carbon atoms in the moiety;

Cycloalkyl is a monocyclic or bicyclic alkyl moiety, having from 3-10 carbon atoms optionally containing 1 to 2 double bonds provided that the moiety is not aromatic, and further provided that the double bonds are not cumulated;

The term “substituted cycloalkyl” is a cycloalkyl group having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;

Alkyl linker means a group selected from alkyl, substituted alkyl, haloalkyl, cycloalkyl, and substituted cycloalkyl having two points of attachment;

The term “heterocycloalkyl”, unless otherwise specified, means a 4 to 8 membered monocylic ring or bicyclic ring, wherein at least one carbon atom is replaced with a heteromember selected from oxygen, nitrogen, —NH—, or —S(O)ₘ— wherein m is zero, 1, or 2, optionally containing from one to three double bonds, provided that the molecule is not aromatic; and provided that ring attachment can occur at either a carbon or nitrogen atom;

The term “substituted heterocycloalkyl” is a heterocycloalkyl group having 1-3 substituents independently selected from halogen, alkyl, substituted alkyl, cycloalkyl, substituted cycloalkyl, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;

Substituted phenyl is a phenyl group having 1-3 substituents independently selected from halogen, alkyl, substituted alkyl, cycloalkyl, substituted cycloalkyl, —OR₅, SR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;

Substituted napthyl is a napthyl group having 1-3 substituents independently selected from halogen, alkyl, substituted alkyl, cycloalkyl, substituted cycloalkyl, —OR₅, SR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;

The term “heteroaryl” means a radical attached via a ring carbon or nitrogen atom of a monocyclic aromatic ring containing five or six ring atoms consisting of carbon and 1, 2, 3, or 4 heteroatoms each selected from the group consisting of non-peroxide O, S, N, with appropriate bonding to satisfy valence requirements as well as a radical (attachment at either carbon or nitrogen) of a fused bicyclic heteroaromatic of about eight to ten ring atoms;

The term “substituted heteroaryl” means a heteroaryl group having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂, phenyl, substituted phenyl, napthyl, substituted napthyl, heteroaryl, and heteroaryl derivatives;

The term “heteroaryl derivatives” means a heteroaryl group having 1-3 substituents independently selected from halogen, —R₅, —OR₅, S(O)ₘR₅, —NR₅R₅, —C(O)R₅, —CN, —C(O)NR₅R₅, —NR₅C(O)R₅, —S(O)₂NR₅R₅, —NR₅S(O)₂R₅, and —NO₂;

Aryl is selected from phenyl, napthyl, substituted phenyl, substituted napthyl, heteroaryl, and substituted heteroaryl derivatives;

Ar is selected from aryl, substituted aryl, and substituted heteroaryl;

The term “aryl cycloalkyl” means a bicyclic ring system containing 9 to 14 carbon atoms wherein one ring is aryl and the other ring is fused to the aryl ring and may be fully or partially saturated in the portion of the ring not fused to the aryl ring, provided that either ring may act as a point of attachment;

The term “substituted aryl cycloalkyl” means an aryl cycloalkyl group having 1-3 substituents independently selected from halogen, —R₅, —OR₆, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;

The term “heteroaryl cycloalkyl” means a bicyclic ring system containing 9 to 14 atoms, wherein one ring is heteroaryl and the other ring is fused to the aryl ring and may be fully or partially saturated in the portion of the ring not fused to the aryl ring, provided that either ring may act as a point of attachment;

The term “substituted heteroaryl cycloalkyl” means a heteroaryl cycloalkyl having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;

The term “aryl heterocycloalkyl” means a bicyclic ring system containing 9 to 14 atoms, wherein one ring is aryl and the other ring is heterocycloalkyl, provided that either ring may act as a point of attachment;

The term “substituted aryl heterocycloalkyl” means an aryl heterocycloalkyl having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂.

The term “heteroaryl heterocycloalkyl” means a bicyclic ring system containing 9 to 14 atoms, wherein one ring is heteroaryl and the other ring is heterocycloalkyl, provided that either ring may act as a point of attachment;

The term “substituted heteroaryl heterocycloalkyl” means an heteroaryl heterocycloalkyl having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;

### DETAILED DESCRIPTION OF THE INVENTION

This invention provides compounds of Formula I as well as stereoisomers and pharmaceutically acceptable salts and prodrugs thereof:

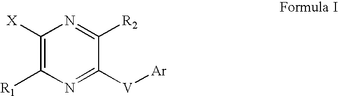

X is selected from —NR₃R₄, —OR₃, —CR₃R₅R₅, —C(O)R₃, —S(O)ₘR₃, —NR₃C(O)R₄, —NR₃S(O)ₘR₄;

V is selected from —O—, —NR₅, or —S(O)ₘ;

m is 0, 1 or 2;

R₁ and R₂ are independently selected from —NH(alkyl), —N(alkyl)₂, —NH(substituted alkyl), —N(substituted alkyl)₂, —O(alkyl), —O(substituted alkyl), halogen, alkyl, substituted alkyl, haloalkyl, cycloalkyl, substituted cycloalkyl, substituted phenyl, naphthyl, substituted naphthyl, heteroaryl, heteroaryl derivatives, substituted aryl, heterocycloalkyl, substituted heterocycloalkyl, substituted heteroaryl, —CR₅R₆Ar, —OAr, —S(O)ₘAr, —NR₅Ar, —S(O)ₘalkyl, —S(O)ₘsubstituted alkyl, —NO₂, —OH, —NH₂, —SH, —C(O)NR₄R₅, —C(S)NR₄R₅, —C(O)NR₅Ar, —S(O)ₘNR5Ar, —NR₅C(O)Ar, —NR₅S(O)nAr, —C(O)Ar, -(alkyl linker)S(O)ₘNH₂, -(alkyl linker)S(O)ₘNR₅Ar, and -(alkyl linker)C(O)Ar;

R₃ and R₄ are independently selected from —H, alkyl, substituted alkyl, haloalkyl, cycloalkyl, substituted cycloalkyl, aryl, heterocycloalkyl, substituted heterocycloalkyl, substituted heteroaryl, aryl cycloalkyl, substituted aryl cycloalkyl, heteroaryl cycloalkyl, substituted heteroaryl cycloalkyl, aryl heterocycloalkyl, substituted aryl heterocycloalkyl, heteroaryl heterocycloalkyl, or substituted heteroaryl heterocycloalkyl;

Each R₅ is independently selected from —H, alkyl, alkylene, alkylyne, cycloalkyl, haloalkyl, and alkyl substituted with 1-3 substituents selected from halogen, —O(alkyl), —NH(alkyl), —N(alkyl)₂, —C(O)NH(alkyl), —C(O)N(alkyl)₂, —NHC(O)alkyl, —N(alkyl)C(O)alkyl, —S(O)ₘalkyl, heterocycloalkyl, substituted heterocycloalkyl and Ar.

Each R₆ is independently selected from alkyl, cycloalkyl, haloalkyl, and alkyl substituted with 1-3 substituents selected from halogen, —O(alkyl), —NH(alkyl), —N(alkyl)₂, —C(O)NH(alkyl), —C(O)N(alkyl)₂, —NHC(O)alkyl, —N(alkyl)C(O)alkyl, —S(O)ₘalkyl, heterocycloalkyl, substituted heterocycloalkyl and Ar;

Halogen is a group selected from —F, —Cl, —Br, —I;

Alkyl means both straight- and branched chain hydrocarbon chains having from 1-10 carbon atoms;

Alkylene means both straight- and branched chain hydrocarbon chains having from 2-10 carbon atoms and a double bond;

Alkylyne means both straight- and branched chain hydrocarbon chains having from 2-10 carbon atoms and a triple bond;

Substituted alkyl is an alkyl moiety from 1-10 carbon atoms having 1-3 substituents independently selected from halogen, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, CN, —NO₂, and Ar;

Haloalkyl is an alkyl moiety having from 1-10 carbon atoms and having 1 to (2v+1) independently selected halogen substituent(s) where v is the number of carbon atoms in the moiety;

Cycloalkyl is a monocyclic or bicyclic alkyl moiety, having from 3-10 carbon atoms optionally containing 1 to 2 double bonds provided that the moiety is not aromatic, and further provided that the double bonds are not cumulated;

The term “substituted cycloalkyl” is a cycloalkyl group having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;

Alkyl linker means a group selected from alkyl, substituted alkyl, haloalkyl, cycloalkyl, and substituted cycloalkyl having two points of attachment;

The term “heterocycloalkyl”, unless otherwise specified, means a 4 to 8 membered monocylic ring or bicyclic ring, wherein at least one carbon atom is replaced with a heteromember selected from oxygen, nitrogen, —NH—, or —S(O)ₘ— wherein m is zero, 1, or 2, optionally containing from one to three double bonds, provided that the molecule is not aromatic; and provided that ring attachment can occur at either a carbon or nitrogen atom;

The term “substituted heterocycloalkyl” is a heterocycloalkyl group having 1-3 substituents independently selected from halogen, alkyl, substituted alkyl, cycloalkyl, substituted cycloalkyl, —OR₅, —S(O)ₘR₅, —NR₅R₅, —C(O)R₅, —CN, —C(O)NR₅R₅, —NR₅C(O)R₅, —S(O)ₘNR₅R₅, NR₅S(O)ₘR₅, and —NO₂;

Substituted phenyl is a phenyl group having 1-3 substituents independently selected from halogen, alkyl, substituted alkyl, cycloalkyl, substituted cycloalkyl, —OR₅, SR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;

Substituted napthyl is a napthyl group having 1-3 substituents independently selected from halogen, alkyl, substituted alkyl, cycloalkyl, substituted cycloalkyl, —OR₅, SR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;

The term “heteroaryl” means a radical attached via a ring carbon or nitrogen atom of a monocyclic aromatic ring containing five or six ring atoms consisting of carbon and 1, 2, 3, or 4 heteroatoms each selected from the group consisting of non-peroxide O, S, N, with appropriate bonding to satisfy valence requirements as well as a radical (attachment at either carbon or nitrogen) of a fused bicyclic heteroaromatic of about eight to ten ring atoms;

The term “substituted heteroaryl” means a heteroaryl group having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂, phenyl, substituted phenyl, napthyl, substituted napthyl, heteroaryl, and heteroaryl derivatives;

The term “heteroaryl derivatives” means a heteroaryl group having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)₂NR₅R₅, —NR₅S(O)₂R₅, and —NO₂;

Aryl is selected from phenyl, napthyl, substituted phenyl, substituted napthyl, heteroaryl, and substituted heteroaryl derivatives;

Ar is selected from aryl, substituted aryl, and substituted heteroaryl;

The term “aryl cycloalkyl” means a bicyclic ring system containing 9 to 14 carbon atoms wherein one ring is aryl and the other ring is fused to the aryl ring and may be fully or partially saturated in the portion of the ring not fused to the aryl ring, provided that either ring may act as a point of attachment;

The term “substituted aryl cycloalkyl” means an aryl cycloalkyl group having 1-3 substituents independently selected from halogen, —R₅, —OR₆, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;

The term “heteroaryl cycloalkyl” means a bicyclic ring system containing 9 to 14 atoms, wherein one ring is heteroaryl and the other ring is fused to the aryl ring and may be fully or partially saturated in the portion of the ring not fused to the aryl ring, provided that either ring may act as a point of attachment;

The term “substituted heteroaryl cycloalkyl” means a heteroaryl cycloalkyl having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;

The term “aryl heterocycloalkyl” means a bicyclic ring system containing 9 to 14 atoms, wherein one ring is aryl and the other ring is heterocycloalkyl, provided that either ring may act as a point of attachment;

The term “substituted aryl heterocycloalkyl” means an aryl heterocycloalkyl having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂.

The term “heteroaryl heterocycloalkyl” means a bicyclic ring system containing 9 to 14 atoms, wherein one ring is heteroaryl and the other ring is heterocycloalkyl, provided that either ring may act as a point of attachment;

The term “substituted heteroaryl heterocycloalkyl” means an heteroaryl heterocycloalkyl having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂.

All temperatures reported herein are in centigrade degrees unless otherwise noted. The term Room Temperature means a temperature between 16° and 25° C.

Compounds provided herein can have one or more asymmetric centers or planes, and all chiral (enantiomeric and diastereomeric) and racemic forms of the compound are included in the present invention. Many geometric isomers of olefins, C═N double bonds, and the like can also be present in the compounds, and all such stable isomers are contemplated in the present invention. Compounds of the invention are isolated in either the racemic form, or in the optically pure form, for example, by resolution of the racemic form by conventional methods such as crystallization in the presence of a resolving agent, or chromatography, using, for example, a chiral HPLC column, or synthesized by a asymmetric synthesis route enabling the preparation of enantiomerically enriched material. The present invention encompasses all possible tautomers of the compounds represented by Formula I. Preferred compounds of this invention include: 3,6-diethyl-N-[(1R,2S)-2-(2-fluoroethoxy)-2,3-dihydro-1H-inden-1-yl]-5-[(4-methylpyridin-2-yl)oxy]pyrazin-2-amine and N-[(1R,2S)-2-ethoxy-2,3-dihydro-1H-inden-1-yl]-3,6-diethyl-5- [(4-methylpyridin-2-yl)oxy]pyrazin-2-amine.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, carbonate salts, and the like salts. Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The expression “prodrug” denotes a derivative of a known direct acting drug, which is transformed into the active drug by an enzymatic or chemical process. Prodrugs of the compounds of formula (I) are prepared by modifying functional groups present on the compound in such a way that the modifications are cleaved, either in routine manipulation or in vivo, to the parent compound. Prodrugs include, but are not limited to, compounds of Formula I wherein hydroxy, amine or sulfhydryl groups are bonded to any group that, when administered to the animal, cleaves to form the free hydroxyl, amino or sulfhydryl group, respectively. Representative examples of prodrugs include, but are not limited to, acetate, formate and benzoate derivatives of alcohol and amine functional groups. See Notari, R. E., “Theory and Practice of Prodrug Kinetics,” Methods in Enzymology, 112:309-323 (1985); Bodor, N., “Novel Approaches in Prodrug Design,” Drugs of the Future, 6(3): 165-182 (1981); and Bundgaard, H., “Design of Prodrugs: Bioreversible-Derivatives for Various Functional Groups and Chemical Entities,” in Design of Prodrugs (H. Bundgaard, ed.), Elsevier, N.Y. (1985).

The invention is illustrated further by the following examples that are not to be construed as limiting the invention in scope or spirit to the specific procedures described in them.

### EXAMPLE 1

The preparation of (1R,2S)-1-({3,6-diethyl-5-[(4-methylpyridin-2-yl)oxy]pyrazin-2-yl}amino)-2,3-dihydro-1H-inden-2-ol (Chart F, Step 3)

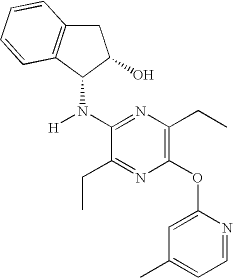

(1R,2S)-1-[(3,6-diethylpyrazin-2-yl)amino]-2,3-dihydro-1H-inden-2-ol (Chart F, Step 1)

A solution of 3-chloro-2,5-diethylpyrazine (171 mg, 1.0 mmol), (1R,2S) -(+)-cis-1-amino-2-indanol (298 mg, 2.0 mmol), tris(dibenzylideneacetone)dipalladium (0) (28 mg, 0.03 mmol), and 2-(di-tertbutylphosphino)biphenyl (18 mg, 0.06 mmol) in toluene (2.0 mL) was purged with nitrogen and treated with sodium t-butoxide (135 mg, 1.4 mmol). The resulting brown suspension was heated to 100° C. for 2 hours. At this time, the reaction was quenched with a saturated water solution of NaHCO₃ and extracted twice with ethyl acetate (20 mL). The combined organics were washed with brine (15 mL), dried over MgSO₄, filtered, and concentrated to give a black solid. This material was purified by Biotage MPLC (40 g column, 25% ethyl acetate/heptane) to afford 184 mg (65%) of (1R,2S)-1-[(3,6-diethylpyrazin-2-yl)amino]-2,3-dihydro-1H-inden-2-ol as a light purple solid. MS (ESI+) for C₁₇H₂₁N₃O m/z 284.0 (M+H)⁺.

(1R,2S)-1-[(3,6-diethyl-5-iodopyrazin-2-yl)amino]-2,3-dihydro-1H-inden-2-ol (Chart F, Step 2)

To a solution of (1R,2S)-1-[(3,6-diethylpyrazin-2-yl)amino]-2,3-dihydro-1H-inden-2-ol (0.58 g, 2.0 mmol) in dimethylsulfoxide (4 mL) was added I₂ (1.02 g, 4.0 mmol). The mixture was stirred at room temperature for 2 days, diluted with EtOAc and sequentially wash with sat. aq. Na₂S₂O₃ and NaHCO₃. The organic extract was dried over MgSO₄, filtered and concentrated. The crude material was purified by Biotage MPLC (90 g column, 20% ethyl acetate/heptane) to afford 0.52 g (63%) of (1R,2S)-1-[(3,6-diethyl-5-iodopyrazin-2-yl)amino]-2,3-dihydro-1H-inden-2-ol as pale yellow solid. MS (ESI+) for C₁₇H₂₀IN₃O m/z 410 (M+H)⁺.

(1R,2S)-1-({3,6-diethyl-5-[(4-methylpyridin-2-yl)oxy]pyrazin-2-yl }amino)-2,3-dihydro-1H-inden-2-ol (Chart F, Step 3)

A vial was charged with (1R,2S)-1-[(3,6-diethyl-5-iodopyrazin-2-yl)amino]-2,3-dihydro-1H-inden-2-ol (100 mg, 0.24 mmol), CuI (4.7 mg, 24 μmol), Cs₂CO₃ (156 mg, 0.48 mmol), and 2-hydroxy-4-methylpyridine (31 mg, 0.29 mmol). The vessel was purged with N₂ and charged with anhydrous DMF (0.24 mL) and dimethylethylenediamine (2.1 mg, 2.6 μL, 24 μmol). The solution was sealed with a teflon cap and heated at 80° C. overnight in a rotating heating block. The mixture was cooled to room temperature, diluted with EtOAc and sequentially washed with water and sat. aq. NaCl. The organic extracts were dried over MgSO₄, filtered and concentrated. This material was purified by Biotage MPLC (90 g column, 20% ethyl acetate/heptane) to afford 41.6 mg (44%) of (1R,2S)-1-({3,6-diethyl-5-[(4-methylpyridin-2-yl)oxy]pyrazin-2-yl}amino)-2,3-dihydro-1H-inden-2-ol as a tan solid. MS (ESI+) for C₂₃H₂₆N₄O₂ m/z 391 (M+H)⁺.

### EXAMPLE 2

The preparation of N-[(1R,2S)-2-ethoxy-2,3-dihydro-1H-inden-1-yl]-3,6-diethyl-5-[(4-methylpyridin-2-yl)oxy]pyrazin-2-amine (Chart F, Step 4)

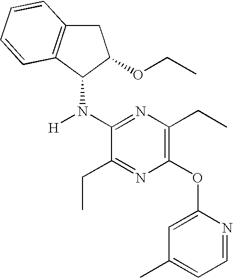

A solution of sodium hydride (60% oil dispersion, 2.8 mg, 0.10 mmol) was suspended in DMF (0.18 mL), purged with nitrogen, and cooled to 0° C. (1R,2S)-1-({3,6-diethyl-5-[(4-methylpyridin-2-yl)oxy]pyrazin-2-yl }amino)-2,3-dihydro-1H-inden-2-ol (18 mg, 46 μmol) with copious gas evolution. The resulting green/golden solution was treated with iodoethane (8 μL, 0.10 mmol) and allowed to warm to room temperature. The mixture was stirred at room temperature overnight and quenched by the addition of water. The mixture was diluted with EtOAc and sequentially washed with water and sat. aq. NaCl, dried over MgSO₄, filtered, and concentrated. This material was purified Biotage MPLC (90 g column, 25% ethyl acetate/heptane) to afford 6.5 mg (34%) of N-[(1R,2S)-2-ethoxy-2,3-dihydro-1H-inden-1-yl]-3,6-diethyl-5-[(4-methylpyridin-2-yl)oxy]pyrazin-2-amine as a yellow oil. MS (ESI+) for C₂₅H₃₀N₄O₂ m/z 419 (M+H)⁺.

### EXAMPLE 3

The preparation of 3,6-diethyl-N-[(1R,2S)-2-(2-fluoroethoxy)-2,3-dihydro-1H-inden-1-yl]-5-[(4-methylpyridin-2-yl)oxy]pyrazin-2-amine (Chart F, Step 4)

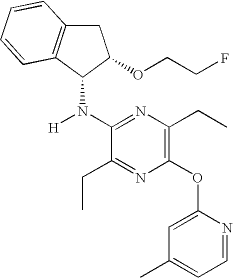

Following the procedure of Example 2 but substituting 2-fluoro-1-iodoethane provided 30 mg (60%) of 3,6-diethyl-N-[(1R,2S)-2-(2-fluoroethoxy)-2,3-dihydro-1H-inden-1-yl]-5-[(4-methylpyridin-2-yl)oxy]pyrazin-2-amine as a yellow oil. MS (ESI+) for C₂₅H₂₉FN₄O₂ m/z 437 (M+H)⁺.

### EXAMPLE 4

The preparation of 3,6-diethyl-N-[(1R,2S)-2-isopropoxy-2,3-dihydro-1H-inden-1-yl]-5-[(4-methylpyridin-2-yl)oxy]pyrazin-2-amine (Chart F, Step 4)

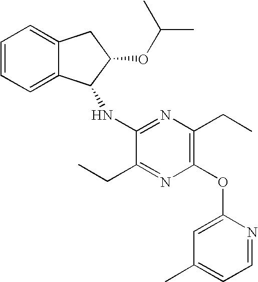

Following the procedure of Example 2 but substituting 2-iodopropane provided 15 mg (34%) of 3,6-diethyl-N-[(1R,2S)-2-isopropoxy-2,3-dihydro-1H-inden-1-yl]-5-[(4-methylpyridin-2-yl)oxy]pyrazin-2-amine as an amber oil. MS (ESI+) for C₂₆H₃₂N₄O₂ m/z 435 (M+H)⁺.

### EXAMPLE 5

The preparation of 3,6-diethyl-5-[(4-methylpyridin-2-yl)oxy]-N-[(1R,2S)-2-propoxy-2,3-dihydro-1H-inden-1-yl]pyrazin-2-amine (Chart F, Step 4)

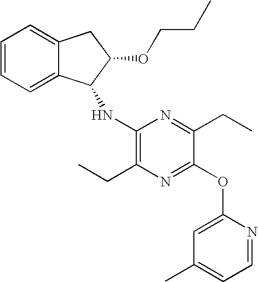

Following the procedure of Example 2 but substituting iodopropane provided 20 mg (45%) of 3,6-diethyl-5-[(4-methylpyridin-2-yl)oxy]-N-[(1R,2S)-2-propoxy-2,3-dihydro-1H-inden-1-yl]pyrazin-2-amine as an amber oil. MS (ESI+) for C₂₆H₃₂N₄O₂ m/z 435 (M+H)⁺.

### EXAMPLE 6

The preparation of (1R,2S)-1-({3,6-diethyl-5-[(4-methylpyridin-2-yl)oxy]pyrazin-2-yl}amino)-2,3-dihydro-1H-inden-2-yl acetate (Chart G, Step 1).

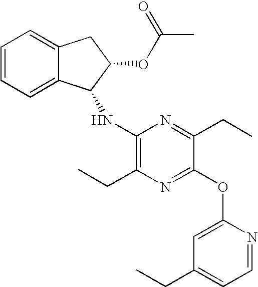

(1R,2S)-1-({3,6-diethyl-5-[(4-methylpyridin-2-yl)oxy]pyrazin-2-yl }amino)-2,3-dihydro-1H-inden-2-ol (0.026 g, 0.065 mmol) was taken up in CH₂Cl₂ (0.7 mL) and charged with pyridine (0.01 mL). The resulting solution was cooled to 0° C. in an ice bath for ten minutes then charged with acetylchloride (0.01 mL) via syringe. After 20 h the reaction was conc. leaving a light yellow semisolid. The crude product was purified via biotage MPLC (25 g column, 1:1:3 EtOAc/CH₂CH₂/heptane) to yield (1R,2S)-1-({3,6-diethyl-5-[(4-methylpyridin-2-yl)oxy]pyrazin-2-yl }amino)-2,3-dihydro-1H-inden-2-yl acetate as an amber oil (0.015 g, 54%). MS (ESI+) for C₂₅H₂₈N₄O₃ m/z 435 (M+H)⁺.

### EXAMPLE 7

The preparation of (1R,2S)-1-({3,6-diethyl-5-[(4-ethylpyridin-2-yl)oxy]pyrazin-2-yl }amino)indan-2-ol (Chart F, Step 3)

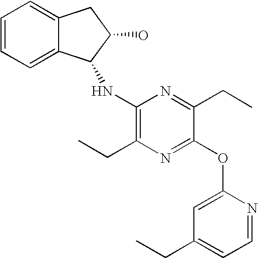

Following the procedure of Example 1 but substituting 4-ethylpyridin-2-ol provided 85 mg (86%) of (1R,2S)-1-({3,6-diethyl-5-[(4-ethylpyridin-2-yl)oxy]pyrazin-2-yl}amino)indan-2-ol as a white solid. MS (ESI+) for C₂₄H₂₈N₄O₂ m/z 406 (M+H)⁺.

### EXAMPLE 8

The preparation of N-[(1R,2S)-2-ethoxy-2,3-dihydro-1H-inden-1-yl]-3,6-diethyl-5-[(4-ethylpyridin-2-yl)oxy]pyrazin-2-amine (Chart F, Step 4)

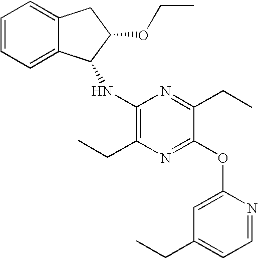

Following the procedure of Example 1 but substituting (1R,2S)-1-({3,6-diethyl-5-[(4-ethylpyridin-2-yl)oxy]pyrazin-2-yl }amino)indan-2-ol provided 21 mg (31%) of N-[(1R,2S)-2-ethoxy-2,3-dihydro-1H-inden-1-yl]-3,6-diethyl-5-[(4-ethylpyridin-2-yl)oxy]pyrazin-2-amine as an amber oil. MS (ESI+) for C₂₆H₃₂N₄O₂ m/z 434 (M+H)⁺.

### EXAMPLE 9

The preparation of (1R,2S)-1-({3,6-diethyl-5-[(3-methylpyridin-2-yl)oxy]pyrazin-2-yl}amino)indan-2-ol (Chart F, Step 3)

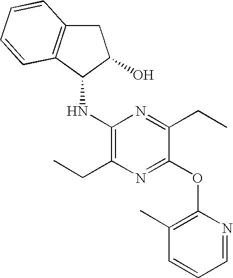

Following the procedure of Example 1 but substituting 3-methylpyridin-2-ol provided 49 mg (54%) of (1R,2S)-1-({3,6-diethyl-5-[(3-methylpyridin-2-yl)oxy]pyrazin-2-yl}amino)indan-2-ol as a beige solid. MS (ESI+) for C₂₃H₂₆N₄O₂ m/z 391 (M+H)⁺.

### EXAMPLE 10

The preparation of N-[(1R,2S)-2-ethoxy-2,3-dihydro-1H-inden-1-yl]-3,6-diethyl-5-[(3-methylpyridin-2-yl)oxy]pyrazin-2-amine (Chart F, Step 4)

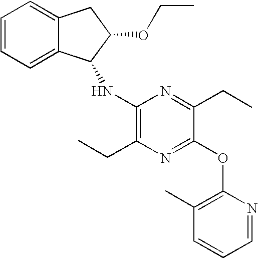

Following the procedure of Example 1 but substituting (1R,2S)-1-({3,6-diethyl-5-[(3-methylpyridin-2-yl)oxy]pyrazin-2-yl }amino)indan-2-ol provided 34 mg (63%) of N-[(1R,2S)-2-ethoxy-2,3-dihydro-1H-inden-1-y]-3,6-diethyl-5-[(3-methylpyridin-2-yl)oxy]pyrazin-2-amine. MS (ESI+) for C₂₅H₃₀N₄O₂ m/z 419 (M+H)⁺.

### EXAMPLE 11

The preparation of (1R,2S)-1-({3,6-diethyl-5-[(5-methylpyridin-2-yl)oxy]pyrazin-2-yl}amino)indan-2-ol (Chart F, Step 3)

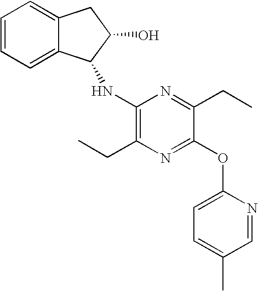

Following the procedure of Example 1 but substituting 5-methylpyridin-2-ol provided 78 mg (82%) of (1R,2S)-1-({3,6-diethyl-5-[(5-methylpyridin-2-yl)oxy]pyrazin-2-yl }amino)indan-2-ol as a beige solid. MS (ESI+) for C₂₃H₂₆N₄O₂ m/z 391 (M+H)⁺.

### EXAMPLE 12

The preparation of N-[(1R,2S)-2-ethoxy-2,3-dihydro-1H-inden-1-yl]-3,6-diethyl-5-[(5-methylpyridin-2-yl)oxy]pyrazin-2-amine (Chart F, Step 4)

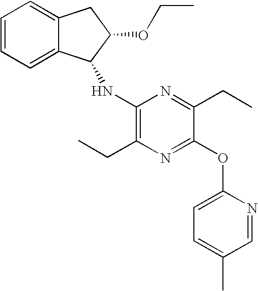

Following the procedure of Example 1 but substituting (1R,2S)-1-({3,6-diethyl-5-[(5-methylpyridin-2-yl)oxy]pyrazin-2-yl }amino)indan-2-ol provided 6 mg (46%) of N-[(1R,2S)-2-ethoxy-2,3-dihydro-1H-inden-1-yl]-3,6-diethyl-5-[(5-methylpyridin-2-yl)oxy]pyrazin-2-amine as an amber oil. MS (ESI+) for C₂₅H₃₀N₄O₂ m/z 419 (M+H)⁺.

### EXAMPLE 13

The preparation of 5-[(4,6-dimethylpyridin-2-yl)oxy]-N-[(1R,2S)-2-ethoxy-2,3-dihydro-1H-inden-1-yl]-3,6-diethylpyrazin-2-amine (Chart F, Step 3)

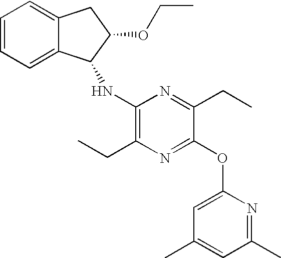

In a 2-necked 25 mL flask under N₂ was added N-[(1R,2S)-2-ethoxy-2,3-dihydro-1H-inden-1-yl]-3,6-diethyl-5-iodopyrazin-2-amine (0.36 g, 0.82 mmol), copper iodide(0.0031 g, 0.016 mmol), cesium carbonate (0.32 g, 0.98 mmol) and 4,6-dimethyl pyridinol (0.12 g, 0.98 mmol). The reaction was heated at 80° C. for 24 hrs. Copper iodide (0.0031 g, 0.016 mmol), cesium carbonate (0.32 g, 0.98 mmol), 4,6 dimethyl pyridinol (0.12 g, 0.98 mmol), N,N′-dimethylethylene diamine (0.0058 g, 0.656 mmol) were added every 24 hr until the reaction was completed. Cool to rt and dilute the reaction mixture with EtOAc, then wash with saturated NaHCO₃ and reextract the aqueous phase with EtOAc (3×40 mL). The EtOAc extract was dried (MgSO₄), filtered, and concentrated in vacuo. The residue was purified by biotage MPLC (40 g column, 10% EtOAc/hexane) to provide 120 mg (34%) of 5-[(4,6-dimethylpyridin-2-yl)oxy]-N-[(1R,2S)-2-ethoxy-2,3-dihydro-1H-inden-1-yl]-3,6-diethylpyrazin-2-amine as a pale yellow oil. . MS (ESI+) for C₂₆H₃₂N₄O₂ m/z 433 (M+H)⁺.

### EXAMPLE 14

The preparation of N-[(1R,2S)-2-ethoxy-2,3-dihydro-1H-inden-1-yl]-3,6-diethyl-5-(3-methylphenoxy)-pyrazin-2-amine (Chart F, Step 3)

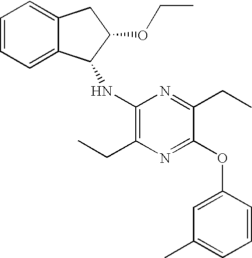

In 2-necked 25 mL flask under N₂ was added N-[(1R,2S)-2-ethoxy-2,3-dihydro-1H-inden-1-yl]-3,6-diethyl-5-iodopyrazin-2-amine (0.13 g, 0.3 mmol), copper iodide(0.0011 g, 0.006 mmol), potassium carbonate (0.05 g, 0.36 mmol), and m-cresol (38 μL, 0.36 mmol). The mixture was heated to 150° C. for 4 hrs. Cool to rt and dilute the reaction mixture with EtOAc, then wash with saturated NaHCO₃ and reextract the aqueous phase with EtOAc (3×40 mL). The EtOAc extract was dried (MgSO₄), filtered, and concentrated in vacuo. The residue was purified by biotage MPLC (40 g column, 5% EtOAc/hexane) to provide 70 mg (56%) of 5-[(4,6-dimethylpyridin-2-yl)oxy]-N-[(1R,2S)-2-ethoxy-2,3-dihydro-1H-inden-1-yl]-3,6-diethylpyrazin-2-amine as pale yellow oil. MS (ESI+) for C₂₆H₃₁N₃O₂ M/z 418 (M+H)⁺.

### EXAMPLE 15

The preparation of 1-({3,6-diethyl-5-[(4-methylphenyl)amino]pyrazin-2-yl}amino)indan-2-ol (Chart F, Step 3).

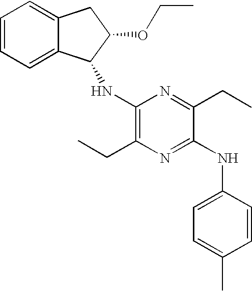

A solution of N-[(1R,2S)-2-ethoxy-2,3-dihydro-1H-inden-1-yl]-3,6-diethyl-5-iodopyrazin-2-amine, 4-methylaniline (2.0 mmol), tris(dibenzylideneacetone)dipalladium (0) (28 mg, 0.03 mmol), and 2-(di-tertbutylphosphino)biphenyl (18 mg, 0.06 mmol) in toluene (2.0 mL) is purged with nitrogen and treated with sodium t-butoxide (135 mg, 1.4 mmol). The resulting suspension is heated to 100° C. for 2 hours. The reaction is quenched with a saturated water solution of NaHCO₃ and extracted twice with ethyl acetate (20 mL). The combined organics are washed with brine (15 mL), dried over MgSO₄, filtered, and concentrated. This material is purified by Biotage MPLC (40 g column, 25% ethyl acetate/heptane) to afford 1-({3,6-diethyl-5-[(4-methylphenyl)amino]pyrazin-2-yl}amino)indan-2-ol.

### EXAMPLE 16

The preparation of N-(2-ethoxy-2,3-dihydro-1H-inden-1-yl)-3,6-diethyl-5-[(4-methylphenyl)thio]pyrazin-2-amine (Chart F, Step 3).

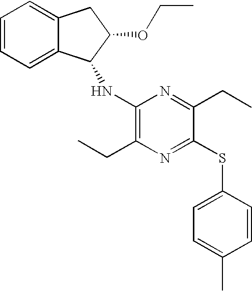

In 2-necked 25 mL flask under N₂ is added N-[(1R,2S)-2-ethoxy-2,3-dihydro-1H-inden-1-yl]-3,6-diethyl-5-iodopyrazin-2-amine (0.3 mmol), copper iodide(0.0011 g, 0.006 mmol), potassium carbonate (0.05 g, 0.36 mmol), and p-thiocresol (0.36 mmol). The mixture is heated to 150° C. for 4 hrs. The cooled reaction mixture is diluted with EtOAc, washed with saturated NaHCO₃, dried (MgSO₄), filtered, and concentrated in vacuo. The residue is purified by biotage MPLC (40 g column, 5% EtOAc/hexane) to provide N-(2-ethoxy-2,3-dihydro-1H-inden-1-yl)-3,6-diethyl-5-[(4-methylphenyl)thio]pyrazin-2-amine.

CRF-R1Receptor Binding Assay for the Evaluation of Biological Activity

The following is a description of the isolation of rat brain membranes for use in the standard binding assay as well as a description of the binding assay itself. It is based on a modified protocol described by De Souza (De Souza, 1987).

To prepare brain membranes for binding assays, rat frontal cortex is homogenized in 10 mL of ice cold tissue buffer (50 mM HEPES buffer pH 7.0, containing 10 mM MgCl₂, 2 mM EGTA, 1 μg/ml aprotinin, 1 μg/ml leupeptin and 1 μg/ml pepstatin). The homogenate is centrifuged at 48,000×g for 10 min. and the resulting pellet re-homogenized in 10 mL of tissue buffer. Following an additional centrifugation at 48,000×g for 10 min., the pellet is resuspended to a protein concentration of 300 μg/mL.

Binding assays are performed in 96 well plates at a final volume of 300 μL. The assays are initiated by the addition of 150 μL membrane suspension to 150 μL of assay buffer containing ¹²⁵I-ovine-CRF (final concentration 150 pM) and various concentrations of inhibitors. The assay buffer is the same as described above for membrane preparation with the addition of 0.1% ovalbumin and 0.15 mM bacitracin. Radioligand binding is terminated after 2 hours at room temperature by filtration through Packard GF/C unifilter plates (presoaked with 0.3% polyethyleneimine) using a Packard cell harvestor. Filters are washed three times with ice cold phosphate buffered saline pH 7.0 containing 0.01% Triton X-100. Filters are assessed for radioactivity in a Packard TopCount.

Alternatively, tissues and cells that naturally express CRF receptors, such as IMR-32 human neuroblastoma cells (ATCC; Hogg et al., 1996), can be employed in binding assays analogous to those described above.

A compound is considered to be active if it has a Kᵢ value of less than about 10 μM for the inhibition of CRF. Nonspecific binding is determined in the presence of excess (10 μM) α-helical CRF.

Inhibition of CRF Stimulated Adenylate Cyclase Activity

Inhibition of CRF-stimulated adenylate cyclase activity can be performed as previously described [G. Battaglia et al., Synapse 1:572 (1987)]. Briefly, assays are carried out at 37° C. for 10 min in 200 mL of buffer containing 100 mM Tris-HCl (pH 7.4 at 37° C.), 10 mM MgCl₂, 0.4 mM EGTA, 0.1% BSA, 1 mM isobutylmethylxanthine (IBMX), 250 units/mL phosphocreatine kinase, 5 mM creatine phosphate, 100 nM guanosine 5′-triphosphate, 100 nM o-CRF, antagonist peptides (various concentrations) and 0.8 mg original wet weight tissue (approximately 40-60 mg protein). Reactions are initiated by the addition of 1 mM ATP/[²P]ATP (approximately 2-4 mCi/tube) and terminated by the addition of 100 mL of 50 mM Tris-HCl, 45 mM ATP and 2% sodium dodecyl sulfate. In order to monitor the recovery of cAMP, 1 mL of [³H]cAMP (approximately 40,000 dpm) is added to each tube prior to separation. The separation of [³²P]cAMP from [³²P]ATP is performed by sequential elution over Dowex and alumina columns.

Alternatively, adenylate cyclase activity can be assessed in a 96-well format utilizing the Adenylyl Cyclase Activation FlashPlate Assay from NEN Life Sciences according to the protocols provided. Briefly, a fixed amount of radiolabeled cAMP is added to 96-well plates that are precoated with anti-cyclic AMP antibody. Cells or tissues are added and stimulated in the presence or absence of inhibitors. Unlabeled cAMP produced by the cells will displace the radiolabeled cAMP from the antibody. The bound radiolabeled cAMP produces a light signal that can be detected using a microplate scintillation counter such as the Packard TopCount. Increasing amounts of unlabeled cAMP results in a decrease of detectable signal over a set incubation time (2-24 hours).

Compounds of the present invention can be prepared using the reactions depicted in the following charts or variations thereof known to those skilled in the art. As illustrated in Chart A, the aminopyrazine A-II can be prepared from the suitably functionalize chloropyrazine A-I (see Chart C) by reaction with the appropriate heterocyclic or carbocyclic amine in the presence of a transition metal catalyst (e.g., palladium(II) acetate or tris(dibenzylideneacetone)dipalladium(0)), base (e.g., sodium or potassium tert-butoxide) in solvents such as but not limited to toluene, DMF, or dioxane (for example, see Buchwald, S. L. J. Org. Chem. 2000, 1158.). A variety of heterocyclic and carbocyclic amines are commercially available or can be synthesized by those skilled in the art. Halogenation of A-II can be accomplished by a number of methods well-known to those skilled in the art utilizing reagents such as N-chlorosuccinimide, N-bromosuccinimide, N-iodosuccinimide, bromine, iodine, pyridinium tribromide in solvents such as dichloromethane, acetic acid, DMF, DMSO etc, to give the halopyrazine A-III. Formation of the claimed compounds I is accomplished by a coupling reaction between A-III and aryl alcohols (for Cul catalysis conditions, see: Buchwald, S. L. J. Am. Chem. Soc. 2002, 124, 7421), anlines under transition metal catalysis (see for example Muci, A. R.; Buchwald, S. L. Topics in Current Chemistry 2002, 219, 131), or aryl thiols (see for example Krinkova, J. Farmaco 2002, 57, 71 and Herradura, P. S.; et al Org. Lett., 2000, 2, 2019). Alternatively, A-I can be coupled with a suitable aryl alcohol, aniline or aryl thiol reagent as described above to provide the arylpyrazine A-IV. Oxidation of the sterically less hindered nitrogen can be effected by using a variety of known oxidizing agents (eg, MCPBA, hydrogen peroxide), and the resulting N-oxide can be treated with phosphorous oxychloride to provide the chloropyrazine A-V. Displacement of the chlorine with a secondary nitrogen as described above provides I.

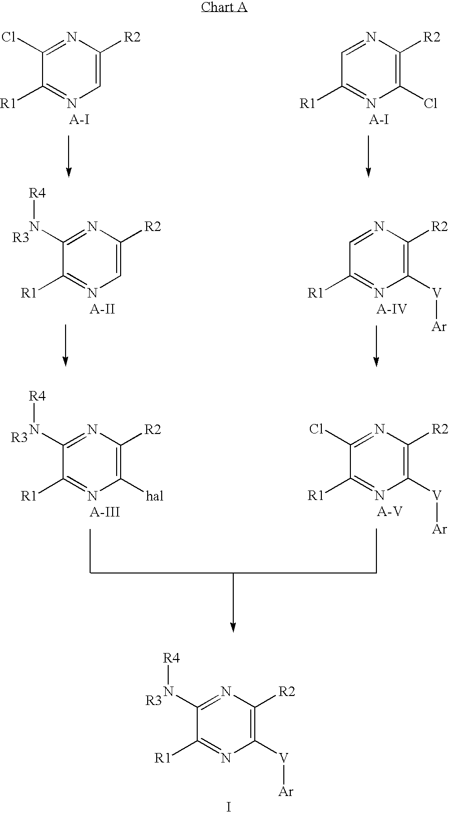

Another way of preparing the compounds of this invention is illustrated in Chart B. Dialkyl-dihalopyrazines B-I (see Chart C) can serve as the starting point for sequential displacement of one chlorine with the appropriate secondary amine (as described in Chart A) followed by reaction at the remaining halogen with a suitable aryl alcohol, aniline or aryl thiol reagent (as described in Chart A) affords I. In some instances, this sequence can be conducted in the opposite order, i.e., reaction with an aryl alcohol, aniline or aryl thiol followed by nucleophilic displacement by a secondary amine.

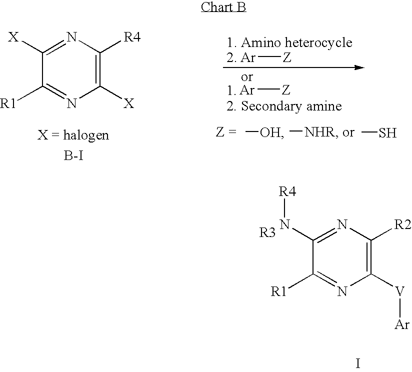

Chart C illustrates the preparation of mono- and dihlopyrazine A-I and B-I respectively when R1 and R4 are alkyl and the same. The reaction sequence shown below follows that described in Chemical and Pharmaceutical Bulletin of Japan, 1979, 27, 2027 when X═Cl.

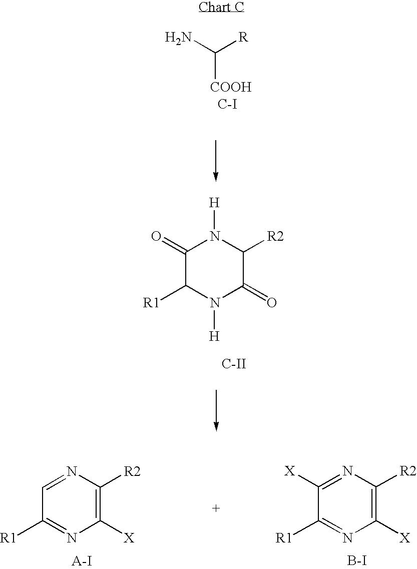

As illustrated in Chart D, treatment of A-V (depicted in Chart A) with an alkoxide or sodium or potassium salt of a thiol should afford compounds such as D-1. Alternatively, if direct alkoxide addition fails, palladium catalysis (see Buchwald, S. L.; et al J. Am. Chem. Soc. 2001, web addition.) or copper catalysis (see Fagan, P. J.; et al J. Am. Chem. Soc. 2000, 122, 5043) of an alkoxide will provide the desired pyrazinyl aryl ether. Another literature method for forming aryl sulfur bonds is demonstrated by the work of Herradura et al. (see, Herradura, P. S.; et al Org. Lett., 2000, 2, 2019).

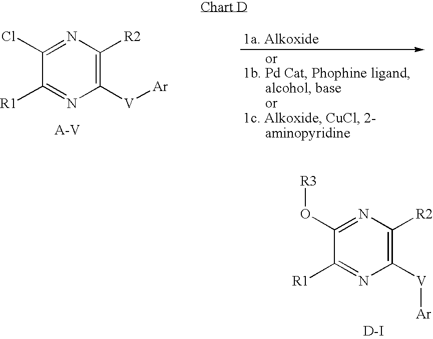

As illustrated in Chart E, treatment of A-V (depicted in Chart A) with a nucleophile such as but not limited to an alkyl Grignard or alkyl lithium reagent would afford compounds such as E-1. Alternatively, treatment with an alkyl boronic acid (see Fu, G. C. et al J. Am. Chem. Soc. 2000, 122, 4020.) under transition metal catalysis should also provide compounds like E-1.

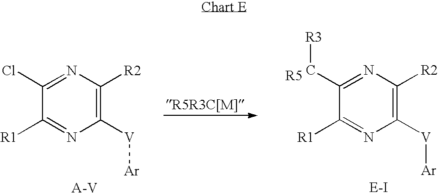

Chart F demonstrates the bets mode for the formation of aryl ethers and anilines. The sequence commences with the coupling of aminoindanol to 2-chloro-3,6-diethylpyrazine under transition metal catalysis to afford F-1. Halogenation with either NBS or I₂ affords F-2. Copper catalyzed coupling to pyridinols provides F-3, while transition metal catalyzed coupling to anilines provides F-4. Alkylation or acylation of F-3 and F-4 provides F-5 and F-6, respectively.

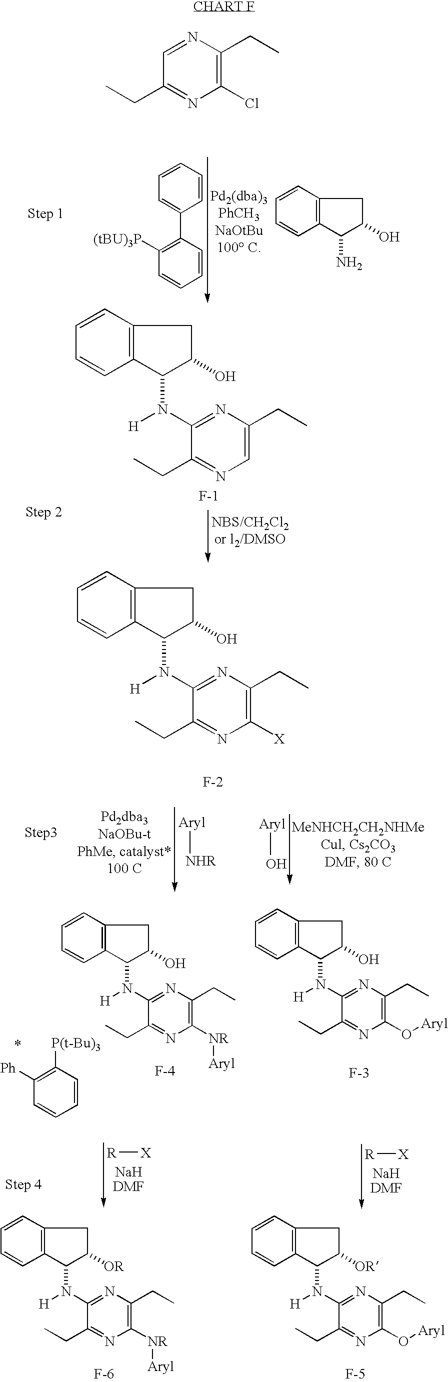

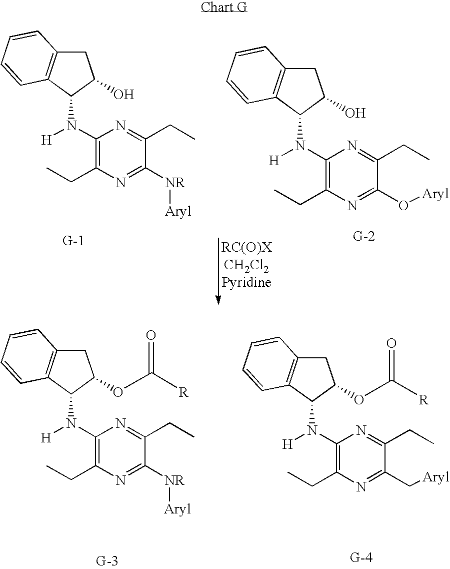

1. A compound of Formula I or a stereoisomer, pharmaceutically acceptable salt, or a prodrug thereof 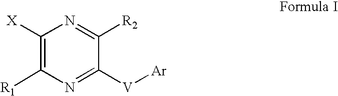
X is selected from —NR₃R₄, —OR₃, —CR₃R₅R₅, —C(O)R₃, —S(O)ₘR₃, —NR₃C(O)R₄, —NR₃S(O)ₘR₄;
V is selected from —O—, —NR₅, or —S(O)ₘ;
m is 0,1 or 2;
R₁ and R₂ are independently selected from —NH(alkyl), —N(alkyl)₂, —NH(substituted alkyl), —N(substituted alkyl)₂, —O(alkyl), —O(substituted alkyl), halogen, alkyl, substituted alkyl, haloalkyl, cycloalkyl, substituted cycloalkyl, substituted phenyl, naphthyl, substituted naphthyl, heteroaryl, heteroaryl derivatives, substituted aryl, heterocycloalkyl, substituted heterocycloalkyl, substituted heteroaryl, —CR₅R₆Ar, —OAr, —S(O)ₘAr, —NR₅Ar, —S(O)ₘalkyl, —S(O)ₘsubstituted alkyl, —NO₂, —OH, —NH₂, —SH, —C(O)NR₄R₅, —C(S)NR₄R₅, —C(O)NR₅Ar, —S(O)ₘNR5Ar, —NR₅C(O)Ar, —NR₅S(O)nAr, —C(O)Ar, -(alkyl linker)S(O)ₘNH₂, -(alkyl linker)S(O)ₘNR₅Ar, and -(alkyl linker)C(O)Ar;
R₃ and R₄ are independently selected from —H, alkyl, substituted alkyl, haloalkyl, cycloalkyl, substituted cycloalkyl, aryl, heterocycloalkyl, substituted heterocycloalkyl, substituted heteroaryl, aryl cycloalkyl, substituted aryl cycloalkyl, heteroaryl cycloalkyl, substituted heteroaryl cycloalkyl, aryl heterocycloalkyl, substituted aryl heterocycloalkyl, heteroaryl heterocycloalkyl, or substituted heteroaryl heterocycloalkyl;
Each R₅ is independently selected from —H, alkyl, alkylene, alkylyne, cycloalkyl, haloalkyl, and alkyl substituted with 1-3 substituents selected from halogen, —O(alkyl), —NH(alkyl), —N(alkyl)₂, —C(O)NH(alkyl), —C(O)N(alkyl)₂, —NHC(O)alkyl, —N(alkyl)C(O)alkyl, —S(O)ₘalkyl, heterocycloalkyl, substituted heterocycloalkyl and Ar:
Each R₆ is independently selected from alkyl, cycloalkyl, haloalkyl, and alkyl substituted with 1-3 substituents selected from halogen, —O(alkyl), —NH(alkyl), —N(alkyl)₂, —C(O)NH(alkyl), —C(O)N(alkyl)₂, —NHC(O)alkyl, —N(alkyl)C(O)alkyl, —S(O)ₘalkyl, heterocycloalkyl, substituted heterocycloalkyl and Ar;
Halogen is a group selected from —F, —Cl, —Br, —I;
Alkyl means both straight- and branched chain hydrocarbon chains having from 1-10 carbon atoms;
Alkylene means both straight- and branched chain hydrocarbon chains having from 2-10 carbon atoms and a double bond;
Alkylyne means both straight- and branched chain hydrocarbon chains having from 2-10 carbon atoms and a triple bond;
Substituted alkyl is an alkyl moiety from 1-10 carbon atoms having 1-3 substituents independently selected from halogen, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, CN, —NO₂, and Ar;
Haloalkyl is an alkyl moiety having from 1-10 carbon atoms and having 1 to (2v+1) independently selected halogen substituent(s) where v is the number of carbon atoms in the moiety;
Cycloalkyl is a monocyclic or bicyclic alkyl moiety, having from 3-10 carbon atoms optionally containing 1 to 2 double bonds provided that the moiety is not aromatic, and further provided that the double bonds are not cumulated;
The term “substituted cycloalkyl” is a cycloalkyl group having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;
Alkyl linker means a group selected from alkyl, substituted alkyl, haloalkyl, cycloalkyl, and substituted cycloalkyl having two points of attachment;
The term “heterocycloalkyl”, unless otherwise specified, means a 4 to 8 membered monocylic ring or bicyclic ring, wherein at least one carbon atom is replaced with a heteromember selected from oxygen, nitrogen, —NH—, or —S(O)ₘ— wherein m is zero, 1, or 2, optionally containing from one to three double bonds, provided that the molecule is not aromatic; and provided that ring attachment can occur at either a carbon or nitrogen atom;
The term “substituted heterocycloalkyl” is a heterocycloalkyl group having 1-3 substituents independently selected from halogen, alkyl, substituted alkyl, cycloalkyl, substituted cycloalkyl, —OR₅, —S(O)ₘR₅, —NR₅R₅, —C(O)R₅, —CN, —C(O)NR₅R₅, —NR₅C(O)R₅, —S (O)ₘNR₅R₅, —NR₅S(O)ₘR₅, and —NO₂;
Substituted phenyl is a phenyl group having 1-3 substituents independently selected from halogen, alkyl, substituted alkyl, cycloalkyl, substituted cycloalkyl, —OR₅, SR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;
Substituted napthyl is a napthyl group having 1-3 substituents independently selected from halogen, alkyl, substituted alkyl, cycloalkyl, substituted cycloalkyl, —OR₅, SR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;
The term “heteroaryl” means a radical attached via a ring carbon or nitrogen atom of a monocyclic aromatic ring containing five or six ring atoms consisting of carbon and 1, 2, 3, or 4 heteroatoms each selected from the group consisting of non-peroxide O, S, N, with appropriate bonding to satisfy valence requirements as well as a radical (attachment at either carbon or nitrogen) of a fused bicyclic heteroaromatic of about eight to ten ring atoms;
The term “substituted heteroaryl” means a heteroaryl group having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂, phenyl, substituted phenyl, napthyl, substituted napthyl, heteroaryl, and heteroaryl derivatives;
The term “heteroaryl derivatives” means a heteroaryl group having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)₂NR₅R₅, —NR₅S(O)₂R₅, and —NO₂;
Aryl is selected from phenyl, napthyl, substituted phenyl, substituted napthyl, heteroaryl, and substituted heteroaryl derivatives;
Ar is selected from aryl, substituted aryl, and substituted heteroaryl;
The term “aryl cycloalkyl” means a bicyclic ring system containing 9 to 14 carbon atoms wherein one ring is aryl and the other ring is fused to the aryl ring and may be fully or partially saturated in the portion of the ring not fused to the aryl ring, provided that either ring may act as a point of attachment;
The term “substituted aryl cycloalkyl” means an aryl cycloalkyl group having 1-3 substituents independently selected from halogen, —R₅, —OR₆, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;
The term “heteroaryl cycloalkyl” means a bicyclic ring system containing 9 to 14 atoms, wherein one ring is heteroaryl and the other ring is fused to the aryl ring and may be fully or partially saturated in the portion of the ring not fused to the aryl ring, provided that either ring may act as a point of attachment;
The term “substituted heteroaryl cycloalkyl” means a heteroaryl cycloalkyl having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂;
The term “aryl heterocycloalkyl” means a bicyclic ring system containing 9 to 14 atoms, wherein one ring is aryl and the other ring is heterocycloalkyl, provided that either ring may act as a point of attachment;
The term “substituted aryl heterocycloalkyl” means an aryl heterocycloalkyl having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂.
The term “heteroaryl heterocycloalkyl” means a bicyclic ring system containing 9 to 14 atoms, wherein one ring is heteroaryl and the other ring is heterocycloalkyl, provided that either ring may act as a point of attachment;
The term “substituted heteroaryl heterocycloalkyl” means an heteroaryl heterocycloalkyl having 1-3 substituents independently selected from halogen, —R₅, —OR₅, —S(O)ₘR₅, —NR₅R₅, —C(O)R₅, —CN, —C(O)NR₅R₅, —NR₅C(O)R₅, —S(O)ₘNR₅R₅, —NR₅S(O)ₘR₅, and —NO₂.

2. A pharmaceutical composition comprising a compound according to claim 1 and at least one pharmaceutically acceptable carrier or excipient.

3. A method for the treatment or prevention of physiological disorders associated with excess of or insufficient amount of CRF, the method comprising administration to a patient in need thereof an effective amount of a compound according to claim 1.

4. A method of inhibiting the binding of CRF to the CRF₁ receptor, the method comprising contacting, in the presence of CRF, a solution comprising a compound of claim 1 with cells expressing the CRF₁ receptor, wherein the compound is present in the solution at a concentration sufficient to reduce levels of CRF binding to IMR32 cells in vitro.

5. A compound according to claim 1 wherein the compound exhibits an IC50 for CRF binding of 1 micromolar or less.

6. A compound according to claim 5 wherein the compound exhibits an IC50 for CRF binding of 100 nanomolar or less.

7. A compound according to claim 6 wherein the compound exhibits an IC50 for CRF binding of 10 nanomolar or less in a standard assay of CRF binding.

8. A method for treating stress, anxiety or depression comprising administering to a patient in need thereof an effective amount of a compound according to claim 1.

9. A compound according to claim 1 wherein V is O.

10. A compound according to claim 1 wherein V is NR₅.

11. A compound according to claim 1 wherein V is S.

12. A compound according to claim 1 wherein Ar is aryl.

13. A compound according to claim 1 wherein Ar is substituted aryl.

14. A compound according to claim 1 wherein Ar is substituted heteroaryl.

15. A compound according to claim 1 selected from the group consisting of
(1R,2S)-1-({3,6-diethyl-5-[(4-methylpyridin-2-yl)oxy]pyrazin-2-yl }amino)-2,3-dihydro-1H-inden-2-ol,
N-[(1R,2S)-2-ethoxy-2,3-dihydro-1H-inden-1-yl]-3,6-diethyl-5-[(4-methylpyridin-2-yl)oxy]pyrazin-2-amine,
3,6-diethyl-N-[(1R,2S)-2-(2-fluoroethoxy)-2,3-dihydro-1H-inden-1-yl]-5-[(4-methylpyridin-2-yl)oxy]pyrazin-2-amine,
3,6-diethyl-N-[(1R,2S)-2-isopropoxy-2,3-dihydro-1H-inden-1-yl]-5-[(4-methylpyridin-2-yl)oxy]pyrazin-2-amine,
3,6-diethyl-5-[(4-methylpyridin-2-yl)oxy]-N-[(1R,2S)-2-propoxy-2,3-dihydro-1H-inden-1-yl]pyrazin-2-amine,
(1R,2S)-1-({3,6-diethyl-5-[(4-methylpyridin-2-yl)oxy]pyrazin-2-yl}amino-2,3-dihydro-1H-inden-2-yl acetate,
(1R,2S)-1-({3,6-diethyl-5-[(4-ethylpyridin-2-yl)oxy]pyrazin-2-yl}amino)indan-2-ol,
N-[(1R,2S)-2-ethoxy-2,3-dihydro-1H-inden-1-yl]-3,6-diethyl-5-[(4-ethylpyridin-2-yl)oxy]pyrazin-2-amine,
(1R,2S)-1-({3,6-diethyl-5-[(3-methylpyridin-2-yl)oxy]pyrazin-2-yl}amino)indan-2-ol,
N-[(1R,2S)-2-ethoxy-2,3-dihydro-1H-inden-1-yl]-3,6-diethyl-5-[(3-methylpyridin-2-yl)oxy]pyrazin-2-amine,
(1R,2S)-1-({3,6-diethyl-5-[(5-methylpyridin-2-yl)oxy]pyrazin-2-yl}amino)indan-2-ol,
N-[(1R,2S)-2-ethoxy-2,3-dihydro-1H-inden-1-yl]-3,6-diethyl-5-[(5-methylpyridin-2-yl)oxy]pyrazin-2-amine,
5-[(4,6-dimethylpyridin-2-yl)oxy]-N-[(1R,2S)-2-ethoxy-2,3-dihydro-1H-inden-1-yl]-3,6-diethylpyrazin-2-amine,
N-[(1R,2S)-2-ethoxy-2,3-dihydro-1H-inden-1-yl]-3,6-diethyl-5-(3-methylphenoxy)-pyrazin-2-amine,
1-({3,6-diethyl-5-[(4-methylphenyl)amino]pyrazin-2-yl}amino)indan-2-ol,
N-(2-ethoxy-2,3-dihydro-1H-inden-1-yl)-3,6-diethyl-5-[(4-methylphenyl)thio]pyrazin-2-amine,
3,6-diethyl-N-[(1R,2S)-2-(2-fluoroethoxy)-2,3-dihydro-1H-inden-1-yl]-5-[(4-methylpyridin-2-yl)oxy]pyrazin-2-amine, and
N-[(1R,2S)-2-ethoxy-2,3-dihydro-1H-inden-1-yl]-3,6-diethyl-5-[(4-methylpyridin-2-yl)oxy]pyrazin-2-amine.
